(19) United States
(12) Patent Application Publication
(10) Pub. No.: US 2011/0008212 A1
(43) Pub. Date: Jan. 13, 2011

(54) CHEMICAL SENSOR HAVING SENSITIVE FILM IMITATING OLFACTORY RECEPTOR

(75) Inventor: NAOYA ICHIMURA, UJI-SHI (JP)

Correspondence Address: DICKSTEIN SHAPIRO LLP, 1633 Broadway, NEW YORK, NY, 10019, US

(73) Assignee: MURATA MANUFACTURING CO., LTD., NAGAOKAKYO-SHI (JP)

(21) Appl. No.: 12/844,251

(22) Filed: Jul. 27, 2010

Related U.S. Application Data
(63) Continuation of application No. PCT/JP2009/050946, filed on Jan. 22, 2009.

(30) Foreign Application Priority Data
Jan. 31, 2008 (JP) 2008-021064

(51) Int. Cl. G01N 30/00; C07K 2/00; B05D 5/12

(52) U.S. Cl. 422/69; 530/300; 427/100

(57) ABSTRACT

A chemical sensor is provided which has an odor detection and discrimination system imparting selectivity and specificity inherent in living organisms for odor substances to a quartz oscillator or a SAW device and which is able to measure a sample in a vapor phase. The chemical sensor includes a sensitive film using a polypeptide prepared by selecting at least part of an amino acid sequence of an olfactory receptor and imitating the at least part thereof so as to have homology thereto. The chemical sensor preferably has a piezoelectric oscillator including the sensitive film.

This is a continuation of application Serial No. PCT/JP2009/050946, filed Jan. 22, 2009, which is hereby incorporated herein by reference. It contains an electronically submitted Sequence Listing which is also hereby incorporated by reference in its entirety. Said ASCII copy, created on Jul. 7, 2010, is named M1071301.txt and is 1,577 bytes in size.

### TECHNICAL FIELD

The present invention relates to a chemical sensor which uses a film obtained by imitating an olfactory receptor responsible for an odor sensing by living organisms, and more particularly relates to a chemical sensor which is used, for example, to measure and discriminate chemical substances and the like in the environment, chemical industries, food administration, and medical testing.

### BACKGROUND ART

The physical oscillation characteristics of a piezoelectric oscillator are influenced by adhesion or adsorption of a substance to the surface thereof, and as a result, the electrical characteristics of the piezoelectric oscillator are changed. By using this phenomenon, a quartz oscillator, a piezoelectric oscillator, is used, for example, as a contamination monitor in a dry process apparatus.

Olfactory cells of an olfactory tissue in living organisms are responsible to detect and discriminate odor substances, and each olfactory cell is each covered with a lipid bilayer membrane; hence, an attempt to imitate an odor discrimination function of the olfactory cell by applying a lipid film on a surface of a quartz oscillator has been carried out. (for example, see “Nioi no Ouyou Kogaku” (Applied Engineering of Odors) by Y. Kurioka, edited by M. Tonoike, published by Asakura Publishing Co., Ltd., p. 210 (Non-Patent Document 1)).

In the attempt described above, the film of a lipid or the like is applied on an electrode surface of the quartz oscillator, and the measurement for detecting a substance is performed using the change in resonant oscillation frequency as an index which is caused by adsorption of a substance to the lipid film. By using many oscillation circuits, it has been studied whether types of adsorbable substances can be discriminated from the adsorption response patterns thereof using the types of lipids to be applied and the difference in characteristics of the substances to be adsorbed.

As disclosed in Japanese Patent No. 3057324 (Japanese Unexamined Patent Application Publication No. 4-121651 (Patent Document 1)), besides the change in resonance frequency caused by the adsorption of substances, it has also been investigated whether by using a quartz oscillator provided with a film formed using a lipid, the impedance and the change in potential of the film are also simultaneously measured to discriminate substances by a smaller number of quartz oscillators.

However, the specificity of the lipid film on the selectivity for odor substances is low, even if a plurality of signals is obtained from the quartz oscillator, the conditional combinations between types and concentrations of odor substances cannot be easily discriminated, and when the odor substances are mixed together, the discrimination thereof is also difficult. In addition, when a plurality of signals is obtained from the quartz oscillator, the electrical circuit for reading the signals becomes complicated, and hence an apparatus therefor becomes expensive.

The presence of proteins binding to odor substances, which are called olfactory receptors, in lipid films of olfactory cells has been disclosed, and the presence of many types of olfactory receptors has also become apparent (for example, Buck, L. B., and Axel, R. (1991). Cell 65, 175-187 (for example, see Non-Patent Document 2)). Amino acid sequences of these many types of olfactory receptors have been identified by analytical studies using gene information, have been recorded in gene databases, such as EMBL and Genbank, and are open to the public on the internet.

By using a recombinant DNA technique, an attempt was carried out to manipulate olfactory receptor genes of mice, and as a result, an olfactory receptor producing an adsorption response to eugenol, i.e., a primary component of herb smells, was identified (for example, see by Kentaro Kajiya et al., (2001) The Journal of Neuroscience, 21(16): 6018 to 6025 (Non-Patent Document 3)).

It is estimated from the characteristics of the amino acid sequence that the conformation of the olfactory receptor of the olfactory cell is the structure in which seven transmembrane α-helices gather together (for example, see Non-Patent Document 2). The olfactory receptor is a high molecular weight membrane protein present in the cell lipid membrane of the olfactory cell and having a molecular weight of 30,000 or more, and it is believed that the olfactory receptor is partly exposed in extracellular water, partly present in the lipid membrane, and partly exposed in an intracellular fluid. Hence, in order to artificially reconstruct the function of the olfactory receptor, the receptor must be partly exposed in water while being allowed to be present in the lipid membrane.

In order to artificially form an olfactory receptor, attempts have been made to express recombinant olfactory receptors in cultured cells or recombinant animals; however, since the amount of olfactory receptor expressed is not large, a functional analysis of the receptor is not easily carried out, and hence, of course, the use thereof is hardly performed. Japanese Unexamined Patent Application Publication No. 5-232006 (Patent Document 2) describes another attempt to use the olfactory receptor, in which after an olfactory receptor is recovered from olfactory tissues of living organisms by extraction and is then applied to a quartz oscillator, the response to a substance is measured.

In the olfactory receptor thus recovered by extraction, all types of olfactory receptors expressed in the whole olfactory tissues are mixed together, and the amounts of the individual olfactory receptors are very small. In addition, components other than the olfactory receptors may also be mixed together, and hence it is difficult to use the olfactory receptor thus obtained for odor discrimination. Furthermore, since it is difficult to determine the purity of the olfactory receptor thus recovered by extraction, a stoichiometric response in which one molecule of the olfactory receptor binds to one molecule of an odor substance cannot be expected even if a predetermined amount thereof is applied to a quartz oscillator.

Besides a specific binding reaction between an odor substance and an olfactory receptor, an antigen-antibody reaction has become well known as a specific binding reaction between biological proteins.

As disclosed in Japanese Patent No. 3892325 (Japanese Unexamined Patent Application Publication No. 2002-350445 (Patent Document 3)), a technique has been investigated in which in order to detect an object substance in a liquid, a composite formed by binding an α-helix polypeptide to an antibody to the object substance functioning as an antigen is applied to a quartz oscillator or a surface acoustic wave (hereinafter referred to as “SAW”) device to form a monomolecular layer. The monomolecular layer is formed to expect structural coloring based on a multilayer thin film interference theory which is the basic coloring principle of the scales of morpho butterfly wings.

According to Patent Document 3, the role of the α-helix polypeptide of the composite is to connect the antibody to the quartz oscillator or the SAW device and to achieve a monomolecular orientation binding to a surface of gold or the like. The antibody enters into a binding reaction with an antigen in an aqueous solution but does not react with a substance in a vapor phase. In addition, since one type of antibody can be produced by immunizing animals with one type of antigen, an extremely large amount of labor is required in order to produce antibodies for various many types of odor substances and hence it is difficult to realize the production of the desired antibodies.

### [Patent Document 1]: Japanese Patent No. 3057324 (Japanese Unexamined Patent Application Publication No. 4-121651

### [Patent Document 2]: Japanese Unexamined Patent Application Publication No. 5-232006

### [Patent Document 3]: Japanese Patent No. 3892325 (Japanese Unexamined Patent Application Publication No. 2002-350445)

[Non-Patent Document 1]: “Nioi no Ouyou Kogaku” (Applied Engineering of Odors) by Y. Kurioka, edited by M. Tonoike, published by Asakura Publishing Co., Ltd., p. 210

### [Non-Patent Document 2]: Buck, L. B., and Axel, R. (1991). Cell 65, 175-187

### [Non-Patent Document 3]: Kentaro Kajiya et al., (2001), The Journal of Neuroscience, 21(16): 6018 to 6025

### DISCLOSURE OF INVENTION

### Problems to be Solved by the Invention

Heretofore, a chemical sensor using a polypeptide prepared by imitating part of the amino acid sequences of receptors in living organisms has not been formed. In addition, it has been difficult to practically use olfactory receptors for odor substances. The reason for this is that when it is intended to use olfactory receptors having selectivity and specificity of living organisms for odor substances, the reconstruction of a high molecular weight membrane protein is difficult to perform on a surface of a device, such as a quartz oscillator. Accordingly, a method for discriminating odor substances using a quartz oscillator or a SAW device, which is provided with the lipid film as described above, has been investigated. However, since the selectivity and specificity of the lipid film for odor substances are low, even if the number of signals from a quartz oscillator is increased to more than one, many sensor elements having different lipid-film components must be inevitably used.

In addition, as described above, the olfactory receptors for odor substances are difficult to be practically used, and according to a related sensor, there has been a problem in that samples in a wet state are only suitable for measurement.

In consideration of the problems described above, an object of the present invention is to provide a chemical sensor which has an odor detection and discrimination system imparting selectivity and specificity inherent in living organisms for odor substances to a quartz oscillator or a SAW device and which is able to measure a sample in a vapor phase as well as a sample in a liquid phase.

### Means for Solving the Problems

In order to solve the above problems, a polypeptide film prepared by imitating part of olfactory receptors is formed on a piezoelectric oscillator, such as a quartz oscillator or a SAW device, to impart selectivity and specificity inherent in living organisms for odor substances thereto in the present invention.

That is, the present invention relates to a chemical sensor which has a sensitive film using at least one polypeptide prepared by selecting at least part of an amino acid sequence of an olfactory receptor and imitating the at least part thereof so as to have homology thereto.

In addition, the chemical sensor of the present invention preferably further has a piezoelectric oscillator including the sensitive film.

In addition, the polypeptide in the chemical sensor of the present invention preferably adsorbs a target molecule in a vapor phase, and the chemical sensor preferably senses a signal from the adsorption between the polypeptide and the target molecule.

The target molecule in the chemical sensor of the present invention is preferably a molecule with which the olfactory receptor including the amino acid sequence imitated by the polypeptide specifically reacts.

In addition, the sequence of the polypeptide in the chemical sensor of the present invention is preferably prepared by comparing the amino acid sequence of the olfactory receptor with an amino acid sequence of another olfactory receptor at estimated corresponding portions thereof and selecting a portion different therefrom.

In the chemical sensor of the present invention, the length of the polypeptide is preferably equal to or less than the length of an α-helix region functioning as a transmembrane region of the amino acid sequence of the olfactory receptor.

The polypeptide preferably includes at least one type of amino acid sequence prepared by imitating one portion or at least two portions of the amino acid sequence of the olfactory receptor, the at least two portions corresponding to amino acid sequences which are not continuously arranged.

In the chemical sensor of the present invention, the polypeptides are preferably arranged so that the relative direction thereof from the N-terminus to the C-terminus coincides with the direction of the olfactory receptor in living organisms, and is preferably used as the sensitive film.

### ADVANTAGES

The present invention can provide a chemical sensor which has an odor detection and discrimination system imparting selectivity and specificity inherent in living organisms for odor substances to a quartz oscillator or a SAW device and which is able to measure a sample in a vapor phase as well as a sample in a liquid phase.

In addition, by performing a reversible reaction, the chemical sensor of the present invention can be continuously used without being thrown away and can also be used in a dry state as a vapor phase sensor as well as in a wet state, and hence the range of use of the chemical sensor can be expanded.

### BRIEF DESCRIPTION OF DRAWINGS

FIG. 1A is a schematic cross-sectional view showing a chemical sensor capable of measuring a response signal to a target molecule using a piezoelectric oscillator (quartz oscillator) which includes sensitive films formed of a polypeptide.

FIG. 1B is an enlarged schematic view of the piezoelectric oscillator shown in FIG. 1A.

FIG. 2 is a graph showing the changes in frequency of quartz oscillators of Example 1 and Comparative Example 1 obtained when eugenol is enclosed in a glass-made container.

FIG. 3 is a graph showing the changes in frequency of the quartz oscillators of Example 1 and Comparative Example 1 obtained when benzene is enclosed in a glass-made container.

FIG. 4 is a graph showing the changes in frequency of the quartz oscillators of Example 1 and Comparative Example 1 obtained when xylene is enclosed in a glass-made container.

FIG. 5 is a graph showing the changes in frequency of quartz oscillators of Examples 1 and 2 obtained when eugenol is enclosed in a glass-made container.

FIG. 6 is a graph showing the change in signal (resonant frequency) of a SAW device obtained when exposure is performed at a eugenol concentration of 0.1 mg/L.

### REFERENCE NUMERALS

1 glass-made container, 2 butyl rubber plug,
3 bottom plate of glass-made container,
4 quartz oscillator, 5 oscillation circuit,
6 power source code, 7 direct current power source,
8 coaxial cable, 9 frequency counter,
20 sensitive film, 21 piezoelectric oscillation plate,
lead electrode, 25 excitation electrode,
30 metal terminal

### BEST MODES FOR CARRYING OUT THE INVENTION

The present invention relates to a chemical sensor which includes a sensitive film formed using a polypeptide which is prepared by selecting at least part of an amino acid sequence of an olfactory receptor and imitating the at least part thereof so as to have homology thereto. For example, when the target molecule specifically binds to the polypeptide, the chemical sensor is able to sense this binding as a signal. As one embodiment of the chemical sensor of the present invention, for example, there may be mentioned a sensor in which a sensitive film including the polypeptide or being formed therefrom is provided on a piezoelectric oscillator or the like.

In this embodiment, the chemical sensor of the present invention includes a sensitive film formed using a polypeptide. The sensitive film is formed of the polypeptide and may optionally include material other than the polypeptide.

The sequence of the polypeptide included in the chemical sensor of the present invention is selected based on the structure of the amino acid sequence of the olfactory receptor. In addition, the homology of the amino acid sequence of the present invention can be measured by using analysis software, and BLAST (Altschl, J, Mol. Biol. 215, 403 to 410 (1990)) may be used as the analysis software. Incidentally, it has been said that when approximately 30% of the sequence of one protein coincides with that of another protein, the proteins show homology to each other.

As for amino acid sequence information of olfactory receptors, desired gene information can be obtained from gene databases open to the public on the internet. As the gene databases, for example, the EMBL database administratively operated by European Molecular Biology Laboratory and the GenBank database provided by National Center of Biotechnology Information may be used. In addition, after a genomic library is formed based on mRNA recovered from olfactory tissues of living organisms, analysis results of genes of the library obtained by using a gene sequence analysis apparatus may also be used.

Hereinafter, the structure of the olfactory receptor will be first described, and a polypeptide to be selected in the present invention will then be described. In addition, a preferable embodiment of a chemical sensor including the polypeptide will be described in detail.

Structure of Olfactory Receptor

An olfactory receptor is a protein including amino acids connected to each other from the N terminus to the C terminus. It is believed that in an olfactory tissue, seven α-helix regions, each functioning as a transmembrane region, form a specific structure. Hereinafter, in this specification, the α-helix region indicates a transmembrane region unless stated otherwise particularly.

According to the structure of the olfactory receptor, the N terminus thereof is exposed to extracellular water outside the cell membrane of an olfactory cell (hereinafter simply referred to as “cell” in some cases), a first α-helix region from the N terminus of the olfactory receptor is present in the cell membrane, and the C terminus side of the first α-helix region is exposed to a cellular fluid inside the cell membrane of the olfactory receptor. A second α-helix region from the N terminus of the olfactory receptor is present in the cell and the cell membrane, and the C terminus side of the second α-helix region is exposed to extracellular water outside the olfactory cell.

A third α-helix region from the N terminus of the olfactory receptor is present outside the cell and in the cell membrane and the C terminus side of the third α-helix region is exposed to the cellular fluid in the olfactory cell. In a fourth α-helix region, the N terminus side thereof is present in the cell and the C terminus side penetrates the cell membrane to be exposed outside the cell; in a fifth α-helix region, the N terminus side thereof is present outside the cell and the C terminus side penetrates the cell membrane to be exposed in the cell; in a sixth α-helix region, the N terminus side thereof is present in the cell and the C terminus side penetrates the cell membrane to be exposed outside the cell; in a seventh α-helix region, the N terminus side thereof is present outside the cell and the C terminus side penetrates the cell membrane to be exposed in the cell; and the C terminus of the olfactory receptor is present in the cell.

These seven α-helix regions gather together on the plane cell membrane of the olfactory receptor so as to function as the receptor.

In these α-helix regions, when the direction of the α-helix regions from the N terminus side to the C terminus side is considered, two adjacent α-helix regions from the N terminus of the olfactory receptor penetrate the cell membrane in directions opposite to each other.

Polypeptide and Sensitive Film

A polypeptide of the present invention has part of the amino acid sequence of the olfactory receptor, and more preferably, has an amino acid sequence of the α-helix regions thereof. After the amino acid sequence of the olfactory receptor is read, an amino acid sequence of a polypeptide of the chemical sensor of the present invention is selected therefrom and is then used. Being artificially and chemically synthesized as a molecule imitating the olfactory receptor, the polypeptide has the selectivity and specificity inherent in living organisms for the target molecule, and hence the selectivity and the specificity for a target molecule are imparted to the chemical sensor including this polypeptide. In addition, the chemical synthesis may be carried out by appropriately using any known method.

The polypeptide is preferably formed from at least one type of amino acid sequence prepared by imitating one portion or at least two portions of the amino acid sequence of the olfactory receptor, where the at least two portions are not continuous arranged amino acid sequences. That is, the polypeptide preferably has an amino acid sequence of the α-helix regions, and any amino acid sequences of the seven α-helix regions may be maintained and used in combination.

In addition, the polypeptide binds to a target molecule in a vapor phase. In this case, the target molecule is, for example, an odor molecule and can be a molecule which is preferably detected by a chemical sensor including this polypeptide.

The target molecule is preferably a molecule with which the olfactory receptor having the amino acid sequence imitated by the polypeptide specifically bonds. The reason for this is that the target molecule can be easily selected, and as a result, the chemical sensor can be easily designed.

As the sequence of the polypeptide, an amino acid sequence of one olfactory receptor and an amino acid sequence of another olfactory receptor are compared with each other at portions estimated to be corresponding portions thereof, and a sequence different from that of the another olfactory receptor may be selected. That is, after amino acid sequences of a plurality of olfactory receptors are compared with each other in parallel to observe the degree of homology therebetween, a portion in which the identical amino acid sequence to that present in a corresponding portion is not present or a portion in which the number of the identical amino acid sequences or amino acids to that present in a corresponding portion is smaller than that therein, that is, a portion having low homology in terms of amino acid sequence, is selected, so that the sequence of the polypeptide of the present invention can be selected. In this case, the portion having low homology in terms of amino acid sequence is assumed as a portion in which at least one base of the amino acid sequence is different from the corresponding portion.

The length of the polypeptide is preferably equal to or less than the length of the α-helix region of the amino acid sequence of the olfactory receptor. A transmembrane α-helix region penetrating the thickness (approximately 5 nm) of the cell membrane of the olfactory receptor has a spiral shape including 3.6 amino-acid residues per turn. Since one amino acid has s thickness of 0.15 nm in a longitudinal direction thereof, the α-helix region is formed of approximately 34 amino acids. Hence, the polypeptide of the present invention is preferably a polypeptide having a an amino acid chain length containing 4 residues or more to 34 residues or less. When the amino acid chain contains 34 residues, the whole α-helix region can be formed. When the amino acid contains 4 residues or more, since it is derived from the α-helix region which has a predetermined structure and which is inherently present in a non-aqueous environmental portion (lipid) of the olfactory receptor, this amino acid can be used for a vapor phase sensor, and the reason for this is that the amino acid is able to react with the target molecule in a dry state.

A plurality of polypeptides, in particular, plural types of polypeptides, are preferably arranged in the sensitive film so that the relative direction from the N terminus to the C terminus is the same as that of the olfactory receptor in living organisms. In particular, polypeptides which include amino acid sequences derived from the odd-numbered α-helix regions from the N terminus of the olfactory receptor, that is, the first α-helix region, the third α-helix region, the fifth α-helix region, and the seventh α-helix region, are arranged so that the directions thereof from the N terminus side to the C terminus side coincide with each other. As is the case described above, polypeptides which include amino acid sequences derived from the even-numbered α-helix regions from the N terminus of the olfactory receptor, that is, the second α-helix region, the fourth α-helix region, and the sixth α-helix region, are arranged so that the directions thereof from the N terminus side to the C terminus side coincide with each other.

The polypeptides including the odd-numbered amino acid sequences and the polypeptides including the even-numbered amino acid sequences are preferably arranged so that the directions from the N-terminus side to the C terminus side are opposite to each other.

It is believed that the olfactory receptor functions with the α-helix regions gathered together on the plane cell membrane, and in the present invention, in order to imitate the conformation of the olfactory receptor, the individual polypeptides are preferably arranged so as not to form spaces therebetween.

### Embodiment 1

The same reference numeral indicates the same or corresponding portion in the drawings of the present invention. The dimensional relationships, such as the length, size, and width, in the drawings are appropriately changed to clarify and simplify the drawings, and actual dimensions are not shown.

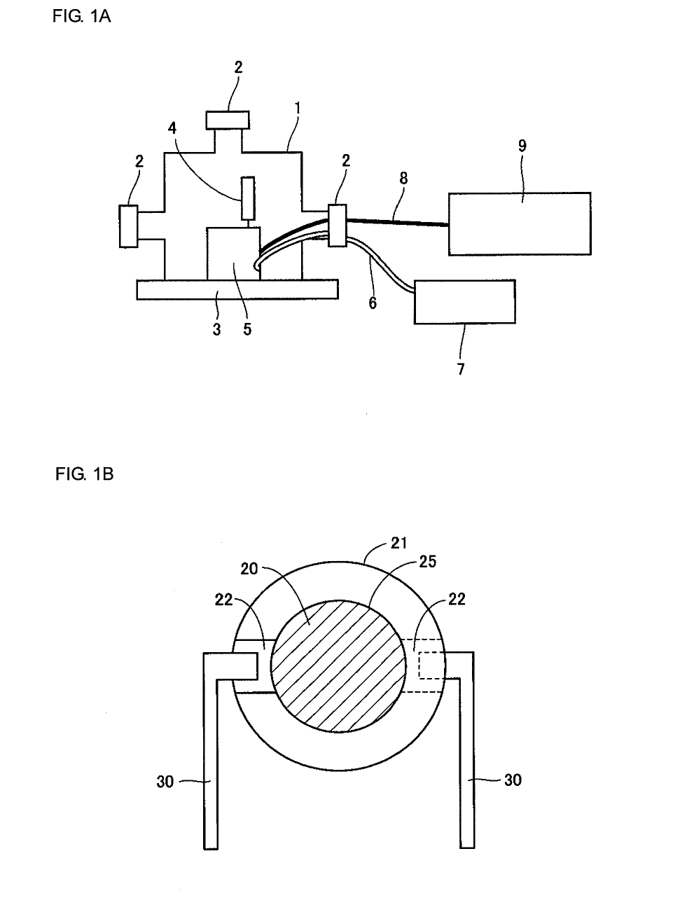

FIG. 1A is a schematic cross-sectional view showing a chemical sensor which uses a piezoelectric oscillator (quartz oscillator) including a sensitive film formed of a polypeptide and which is able to measure a response signal to a target molecule. FIG. 1B is an enlarged schematic view of the piezoelectric oscillator shown in FIG. 1A.

Hereinafter, the chemical sensor will be described with reference to FIGS. 1A and 1B.

A glass container 1 has tube portions to be capped with butyl rubber plugs 2 which can tightly seal the inside of the glass container 1 in combination with a bottom plate 3 thereof. In the glass container 1, a quartz oscillator 4 functioning as a piezoelectric oscillator and an oscillation circuit 5 exciting this quartz oscillator 4 are present. In addition, electrical power is supplied from a direct current power source 7 to the oscillation circuit 5 through a power source connector 6 penetrating the butyl rubber plug 2. The resonant frequency signal of the quartz oscillator 4 is transmitted from the oscillation circuit 5 to a frequency counter 9 through a coaxial cable 8 penetrating the butyl rubber plug 2, so that the resonant frequency of the quartz oscillator 4 can be measured. The oscillation circuit 5 can simultaneously excite a quartz oscillator 4 having a film formed of a polypeptide and a quartz oscillator 4 having a film formed of a β-mercaptoethanol.

The quartz oscillator 4 includes sensitive films 20 formed of a polypeptide on excitation electrodes 25 provided on two surfaces of a piezoelectric oscillation plate 21. The excitation electrodes 25 are each electrically connected to the oscillation circuit 5 by a lead electrode 22 and a metal terminal 30.

In this embodiment, a polypeptide is formed by imitating the directionality from the N terminus side to the C terminus side of the α-helix regions in the cell membrane of the olfactory receptor, so that the selectivity and specificity inherent in living organisms for an odor substance (target molecule) can be imparted to the quartz oscillator (or a SAW device).

As the polypeptide of this embodiment, the polypeptide described above may be used.

It has been well known that an alkane compound containing a thiol group easily forms a covalent bond with gold and forms a monomolecular film on the surface thereof. A reaction between gold and the thiol group may be used in this embodiment, and for forming a film on an electrode surface of a quartz oscillator or a SAW device using a polypeptide, a method of binding a thiol compound to a polypeptide may be used.

The reaction between a thiol and a polypeptide can be easily carried out when gold is used for the electrode surface of a quartz oscillator or SAW device; however, a noble metal other than gold, such as silver, may also be used when conditions are properly adjusted.

As the method to bind a thiol compound to a polypeptide, for example, a method may be carried out by using a compound, such as 8-amino-1-octanethiol, containing both an amino group and a thiol group in the same molecule, and the thiol is first allowed to react with gold on an electrode surface to form a monomolecular film so as to introduce the amino group on the electrode surface, and by using a compound, such as a N-hydroxysuccimide ester, e.g., disuccinimidyl suberate (DSS), having two reactive portions which react with an amino group, to form a covalent bond, the amino group on the electrode surface binds to a polypeptide having an amino group.

Alternatively, the polypeptide used for forming the film may be formed in such a way that after a polypeptide is formed by binding an amino acid having a thiol moiety, such as cysteine, to the N-terminus or the C terminus of a polypeptide having an amino acid sequence of transmembrane α-helix portions of the olfactory receptor to a compound having a thiol group and an amino group, the thiol is allowed to react with a gold electrode surface to introduce the amino group on the electrode surface, and by using a compound having a reactive portion, such as an N-hydroxysuccinimide ester, e.g., such as sulfosuccinimidyl-4-(N-maleimidomethyl)cyclohexane-1-carboxylate, to form a covalent bond by a reaction with an amino group and a reactive portion, such as a maleimide, to form a covalent bond by a reaction with a thiol group, the amino group on the electrode surface is made to bind to the polypeptide having an amino acid having a thiol group at the terminus thereof such as cysteine. However, the method is not limited to those described above.

Instead of using the reaction between gold and the thiol sulfur in order to form a film capable of imparting selectivity and specificity on an electrode surface of a quartz oscillator or a SAW device by using a polypeptide synthesized by imitating the olfactory receptor, for example, other known methods may be used which can be easily performed by a person skilled in the art, such as an alkylation method, a diazo method, a thiol disulfide formation reaction, a Schiff base formation method, a chelate bonding method, a tosyl chloride method, and biotin-avidin bond formation, in which a covalent bond is formed by introducing a functional group, such as an amino group or a thiol group, on an electrode surface of a quartz oscillator or a SAW device and binding the functional group to a polypeptide. In addition, the known methods and the above-described methods may be appropriately selected and may be performed in combination.

It is believed that the olfactory receptor functions since the transmembrane α-helices gather together on a plane cell membrane, and in the present invention, in order to imitate the above structure, the compound having a thiol group and an amino group is preferably reacted with the electrode surface of a quartz oscillator or a SAW device so as not to form spaces between polypeptides.

In the present invention, as a process to change the space between polypeptides on the electrode surface in a process for forming a monomolecular film on the electrode surface, a compound having a thiol and a hydroxyl group, such as β-mercaptoethanol, or a compound having only a thiol group as a functional group, such as octanethiol, can be mixed with a compound having a thiol and an amino group, such as 8-amino-1-octanethiol, to react therewith, so that the response characteristics on the electrode surface can be changed.

According to this embodiment, when the film is formed on a piezoelectric oscillator by imitating the olfactory receptor responsible for olfactory characteristics of living organisms, a chemical sensor for odor substances can be formed which imitates the olfaction of living organisms. By using this chemical sensor, various target substances, such as living odors, perfume, smell substances, aromatic substances, medicines, and food components, can be detected and in addition, the function thereof can be easily installed in electronic circuits and electronic apparatuses.

Discrimination of odor substances, which has been the subject of related techniques, can be performed using a small number of elements, and the elements can be driven, for example, by a simple excitation circuit; hence, a circuit structure measuring a plurality of electrical characteristics is not required.

Hereinafter, the present invention will be described in detail with reference to Examples; however, the present invention is not limited thereto.

### EXAMPLES

### Example 1

As a gene coding for an amino acid sequence (hereinafter referred to as “amino acid sequence A”) of an olfactory receptor used in this example, information on File ID No. AB061228 was obtained from the EMBL database. Simultaneously, as a gene coding for an amino acid sequence (hereinafter referred to as “amino acid sequence B”) of an olfactory receptor different from the above olfactory receptor, information on File ID No. AY317355 was obtained from the EMBL database. Subsequently, the sequence of File ID No. AB061228 and the sequence of File ID No. AY317355 were compared with each other, and it was confirmed that the olfactory receptor having an amino acid sequence coded by File ID No. AB061228 responded to eugenol, i.e., a primary component of flavor of cumin which is one type of herb.

As the sequence of the polypeptide used in this example, the amino acid sequence A of the olfactory receptor and the amino acid sequence B of the different olfactory receptor were compared with each other at estimated corresponding portions thereof, and a sequence of the amino acid sequence A different from that of the amino acid sequence B was selected. That is, after amino acid sequences of a plurality of olfactory receptors were compared with each other in parallel to observe the degree of homology therebetween, a portion in which the identical amino acid sequence to that present in a corresponding portion was not present or a portion in which the number of the identical amino acid sequences or amino acids to that present in a corresponding portion is small, that is, a portion having low homology in terms of the amino acid sequence the portion having low homology having an amino acid sequence portion in which at least one base thereof is different from the corresponding portion in this case), was selected, so that the sequence of the polypeptide of the present invention was selected.

In this example, it was confirmed that between “LFVFATFNEISTLLI (SEQ ID No. 3)” which is an amino acid sequence of 15 residues from the 200th residue starting from the N-terminus of the amino acid sequence A and “MGVANVIFLGVPVLF (SEQ ID No. 4) which is an amino acid sequence of 15 residues from the 200th residue starting from the N-terminus of the amino acid sequence B, only 2 out of 15 residues were identical, and 13 residues were not identical.

Furthermore, it was also confirmed that between “ITIFHGTILFLY (SEQ ID No. 5)” which is an amino acid sequence of 12 residues from the 249th residue starting from the N-terminus of the amino acid sequence A and “VIIFYGTILFMY (SEQ ID No. 6) which is an amino acid sequence of 12 residues from the 249th residue starting from the N-terminus of the amino acid sequence B, 8 out of 12 residues were identical, and 4 residues were not identical.

Subsequently, the transmembrane α-helix regions were estimated in the amino acid sequence A coded by File ID No. ABO61228, from an estimated structure of the olfactory receptor. It was estimated that an amino acid sequence in the vicinity of the 200th amino acid starting from the N-terminus corresponded to a fifth transmembrane region from the N-terminus. It was estimated that an amino acid sequence in the vicinity of the 250th amino acid starting from the N-terminus corresponded to a sixth transmembrane region from the N-terminus. In the amino acid sequence B coded by File ID No. AY317355, it was estimated that an amino acid sequence in the vicinity of the 200th amino acid starting from the N-terminus corresponded to a fifth transmembrane region from the N-terminus, and that an amino acid sequence in the vicinity of the 250th amino acid starting from the N-terminus corresponded to a sixth transmembrane region from the N-terminus.

Accordingly, a polypeptide composed of “LFVFATFNEISTLLI (SEQ ID No. 3)” which was the amino acid sequence of 15 residues from 200th residue staring from the N-terminus of the amino acid sequence A and a polypeptide composed of “ITIFHGTILFLY (SEQ ID No. 5)” which was the amino acid sequence of 12 residues from 249th residue starting from the N-terminus of the amino acid sequence A were selected as polypeptides forming a film on a quartz oscillator.

In addition, in order to arrange the C-terminus side of the polypeptide composed of “LFVFATFNEISTLLI (SEQ ID No. 3)” which is an amino acid sequence of 15 residues from 200th residue starting from the N-terminus of the amino acid sequence A at the electrode surface side so that the orientation of the polypeptide formed into the film coincides with that of the olfactory receptor, a cysteine residue was added to the C-terminus to form a sequence of “LFVFATFNEISTLLIC (SEQ ID No. 1)” (hereinafter referred to as “polypeptide 1), and by using a peptide solid phase synthesis method (Fmoc synthesis method), polypeptide synthesis was carried out. In order to arrange the N-terminus side the polypeptide composed of “ITIFHGTILFLY (SEQ ID No. 5)” which is an amino acid sequence of 12 residues from 249th residue starting from the N-terminus of the amino acid sequence A at the electrode surface side, a cysteine residue was added to the N-terminus to as to form a sequence of “CITIFHGTILFLY (SEQ ID No. 2)” (hereinafter referred to as “polypeptide 2), and as in the case described above, polypeptide synthesis was carried out.

| Polypeptide 1: | |
|---|---|
| “LFVFATFNEISTLLIC | (SEQ ID No. 1)” |
| Polypeptide 2: | |
| “CITIFHGTILFLY | (SEQ ID No. 2)” |

A plasma treatment was performed on a gold electrode surface of a quartz oscillator having resonant frequency characteristics at 24 MHz, so that organic substances, sulfur compounds, and the like, adhered on the gold electrode surface were removed. Next, an alkanethiol solution in which 8-amino-1-octanethiol was dissolved in ethanol at a concentration of 1 mM was prepared. The quartz oscillator processed by the plasma treatment was immediately dipped into the alkanethiol solution and was held for 30 minutes at room temperature.

After the alkanethiol solution was removed from the quartz oscillator, the electrode surface of the quartz oscillator was washed three times with ethanol. After the washing was finished, ethanol was removed from the quartz oscillator, and the electrode surface was washed three times with sterilized distilled water.

Next, a solution was prepared in which sulfosuccinimidyl-4-(N-maleimidomethyl)cyclohexane-1-carboxylate was dissolved in PBS (0.1 M-NaPO₄, 0.15 M-NaCl, 2 mM-EDTA, pH 7.2) which was a buffer solution containing phosphoric acid and a normal saline component to have a concentration of 50 μl. Subsequently, the quartz oscillator was dipped into the above solution and was held for 3 hours at room temperature. The reaction solution was removed from the quartz oscillator, and washing was performed three times with PBS.

After the synthesized polypeptides 1 and 2 were dissolved in dimethyl sulfoxide, the cysteine was reduced by using tris(2-carboxyethyl)phosphine as the reducing agent, and a polypeptide solution was prepared using PBS so that the polypeptides each had a concentration of 100 μM. The quartz oscillator which reacted with sulfosuccinimidyl-4-(N-maleimidomethyl)cyclohexane-1-carboxylate and then washed as described above was dipped into the polypeptide solution and was held for 6 hours at room temperature.

Subsequently, after the polypeptide solution was removed, the quartz oscillator was washed three times with PBS and then washed three times with ethanol. After ethanol was removed, washing was performed three times with sterilized distilled water, and air drying was performed at room temperature, so that a quartz oscillator provided with a film including the polypeptides 1 and 2 was formed.

### Comparative Example 1

After a quartz oscillator processed by a plasma treatment was dipped into a solution in which β-mercaptoethanol was dissolved in ethanol to have a concentration of 1 mM instead of using 8-amino-1-octanethiol for 30 minutes at room temperature, an electrode surface of the quartz oscillator was washed three times with ethanol and was then washed three times with sterilized distilled water, and the quartz oscillator thus obtained was used for an experiment as a reference.

Evaluation Method

The quartz oscillator of Example 1 and the quartz oscillator of Comparative Example 1 were each connected to an oscillation circuit using an inverter IC, and the oscillation frequency of each quartz oscillator was measured by using a frequency counter. The detailed measurement method will be described with reference to FIGS. 1A and 1B.

A glass container 1 had tube portions to be capped with butyl rubber plugs 2 which could tightly seal the inside of the glass container 1 in combination with a bottom plate 3 thereof. In the glass container 1, a quartz oscillator 4 functioning as a piezoelectric oscillator and an oscillation circuit 5 exciting this quartz oscillator 4 were placed. Electrical power was supplied from a direct current power source 7 to the oscillation circuit 5 through a power source cord 6 penetrating the butyl rubber plug 2. A resonant frequency signal of the quartz oscillator 4 was transmitted from the oscillation circuit 5 to a frequency counter 9 through a coaxial cable 8 penetrating a butyl rubber plug 2, so that the resonant frequency of the quartz oscillator 4 was measured.

The quartz oscillator 4 included sensitive films 20 formed of a polypeptide on excitation electrodes 25 formed of gold electrodes provided on two surfaces of a piezoelectric oscillation plate 21.

An odor substance was inserted into the glass-made container 1 through a butyl rubber plug 2 using a syringe barrel and a syringe needle. Subsequently, the quartz oscillator of Example 1 was connected inside the glass-made container 1, and electrical power was supplied to the oscillation circuit 5, so that the quartz oscillator 4 was excited.

Next, eugenol was introduced in the glass container 1 to a concentration of 1 mg/L, and the change in resonant frequency of the quartz oscillator caused by this incorporation was measured by a frequency counter. Eugenol is a compound having an aromatic ring structure in the molecule.

Evaluation Result 1

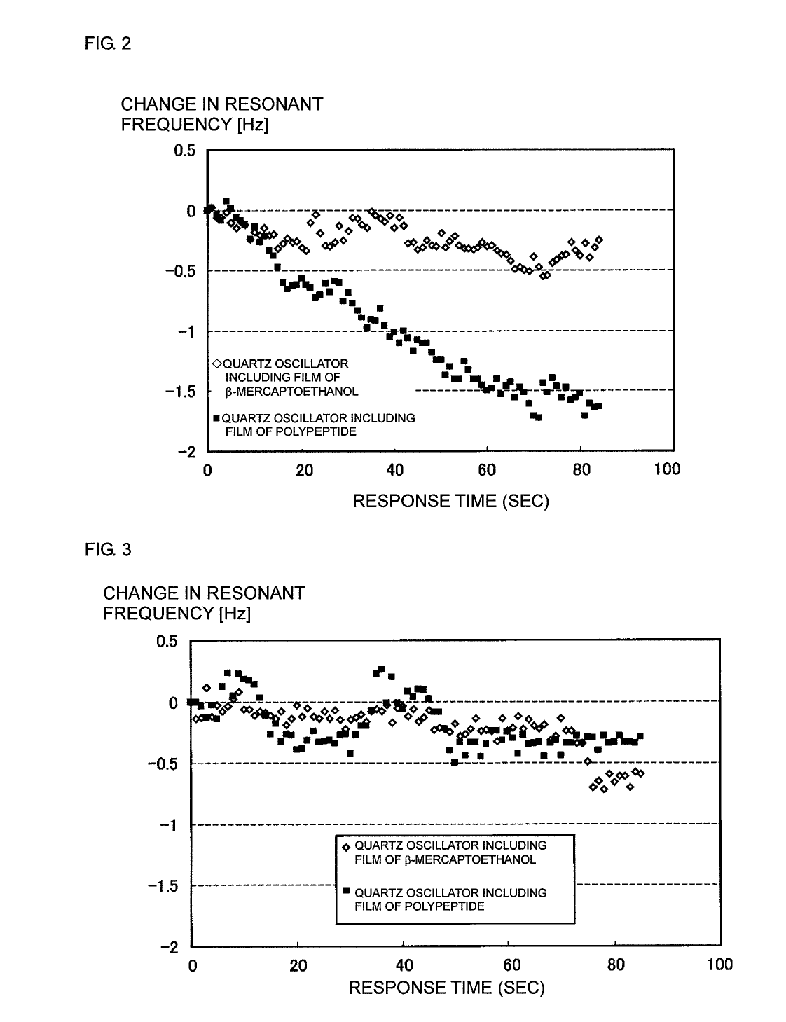

FIG. 2 shows the change in frequency of the quartz oscillator obtained when eugenol was in the glass container 1. The horizontal axis indicates the time from the introduction (response time), and the vertical axis indicates resonant frequency (change in resonant frequency). In addition, the outline dot indicates the result of Comparative Example 1, and the black dot indicates the result of Example 1.

It was found that by the introduction of eugenol, the resonant frequency of the quartz oscillator of Example 1 was gradually decreased with the response time. This indicates that eugenol caused an adsorption reaction on the electrode surface of the quartz oscillator of Example 1. On the other hand, the resonant frequency of the quartz oscillator of Comparative Example 1 was not changed by the eugenol. This indicates that eugenol caused no adsorption reaction on the electrode surface of the quartz oscillator of Comparative Example 1.

Evaluation Result 2

Although eugenol is a compound having an aromatic ring structure in the molecule, benzene and xylene are also compounds having an aromatic ring structure, and have a degree of similarity to eugenol in view of a chemical structural. Hence, benzene and xylene were each added to the glass container 1 to a concentration of 1 mg/L, and the change in resonant frequency of the quartz oscillator caused by this addition was measured by a frequency counter.

FIG. 3 shows the change in frequency of the quartz oscillator obtained when benzene was enclosed in the glass-made container 1. The horizontal axis indicates the time from the addition (response time), and the vertical axis indicates resonant frequency (change in resonant frequency). In addition, the outline dot indicates the result of Comparative Example 1, and the black dot indicates the result of Example 1.

As shown in FIG. 3, the resonant frequency of each of the quartz oscillators of Example 1 and Comparative Example 1 showed no change in resonant frequency caused by the benzene. This indicates that benzene caused no adsorption reaction on the electrode surface of each of the quartz oscillators of Example 1 and Comparative Example 1.

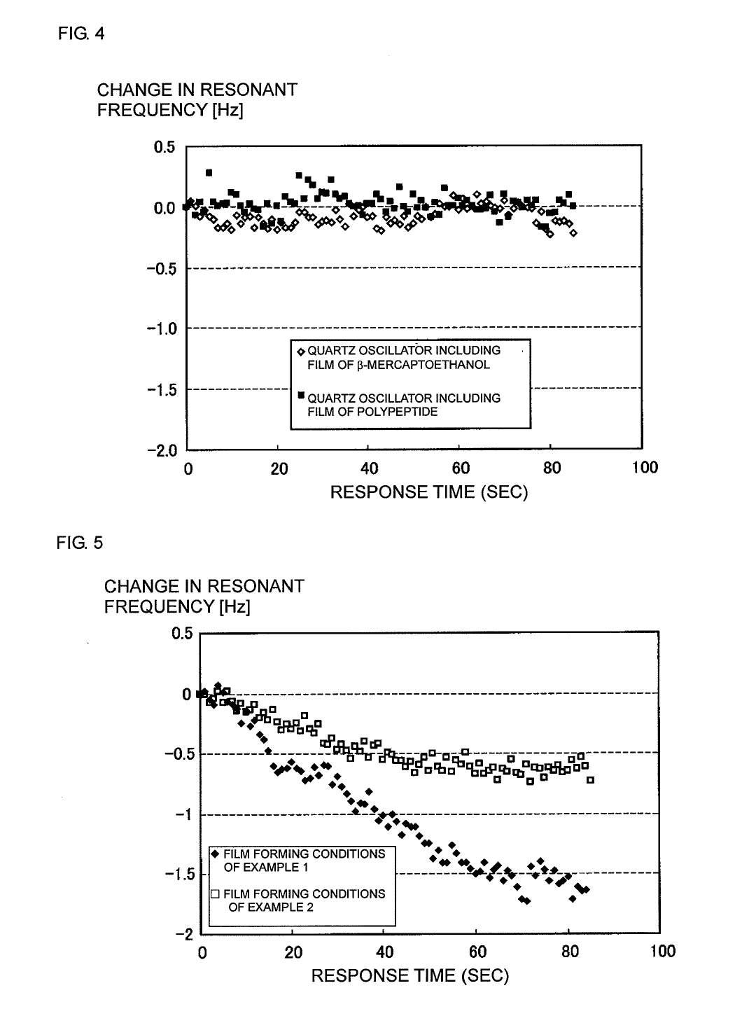

FIG. 4 shows the change in frequency of the quartz oscillator obtained when xylene was added to the glass container 1. The horizontal axis indicates the time from the addition (response time), and the vertical axis indicates resonant frequency (change in resonant frequency). In addition, the outline dot indicates the result of Comparative Example 1, and the black dot indicates the result of Example 1.

As shown in FIG. 4, the resonant frequency of each of the quartz oscillators of Example 1 and Comparative Example 1 showed no change in resonant frequency caused by the xylene. This indicates that xylene caused no adsorption reaction on the electrode surface of each of the quartz oscillators of Example 1 and Comparative Example 1.

From the results shown in FIGS. 2 to 4, it was shown that the quartz oscillator of Example 1 caused an adsorption reaction with eugenol and produced no response to benzene and xylene each of which has an aromatic ring structure similar to that of eugenol.

It was thus shown that in the quartz oscillator in which the film was formed of the polypeptide synthesized in such a way that by using the amino acid sequence of the olfactory receptor for eugenol, the transmembrane region, which is a part of the above amino acid sequence, was imitated, an adsorption response only to eugenol could be produced, and eugenol could be discriminated from benzene and xylene similar thereto.

### Example 2

Instead of dipping the quartz oscillator processed by a plasma treatment of Example 1 into the alkanethiol solution, the quartz oscillator processed by a plasma treatment was dipped into a different solution and was held for 30 minutes at room temperature. The different solution was prepared by mixing 8-amino-1-octanethiol and β-mercaptoethanol at an equal mole ratio so that each had a concentration 0.5 mM.

Steps were performed in a manner similar to that in Example 1 except for the step described in the preceding paragraph. Accordingly, a quartz oscillator of Example 2 was formed.

The difference in response to eugenol between the quartz oscillators of Examples 1 and 2 was measured and evaluated. The measurement was performed in a manner similar to the method described above.

Evaluation Result 3

FIG. 5 shows the change in frequency of the quartz oscillator obtained when eugenol was in the glass container 1. The horizontal axis indicates the time from the addition (response time), and the vertical axis indicates resonant frequency (change in resonant frequency). The outline dot indicates the result of Example 2, and the black dot indicates the result of Example 1.

It was found that by the addition of eugenol, the resonant frequency of the quartz oscillator of Example 2 gradually decreased as the response time increased. However, compared with the change in resonant frequency of the quartz oscillator of Example 1, the change was slow. Accordingly, it was shown that the response characteristics of the quartz oscillator of Example 1 were different from that of the quartz oscillator of Example 2 and that depending on component conditions of an alkanethiol for the gold electrode, the response characteristics of the quartz oscillator could be changed.

### Example 3

By using the film forming method of the polypeptides 1 and 2 for the quartz oscillator of Example 1 as described above, a film of the polypeptides 1 and 2 was formed on a SAW device. The response to eugenol of the SAW device provided with the film was measured and confirmed.

As the oscillation frequency of the SAW device, signals obtained by dividing the oscillation frequency of the oscillation circuit were measured by a frequency counter.

Evaluation Result 4

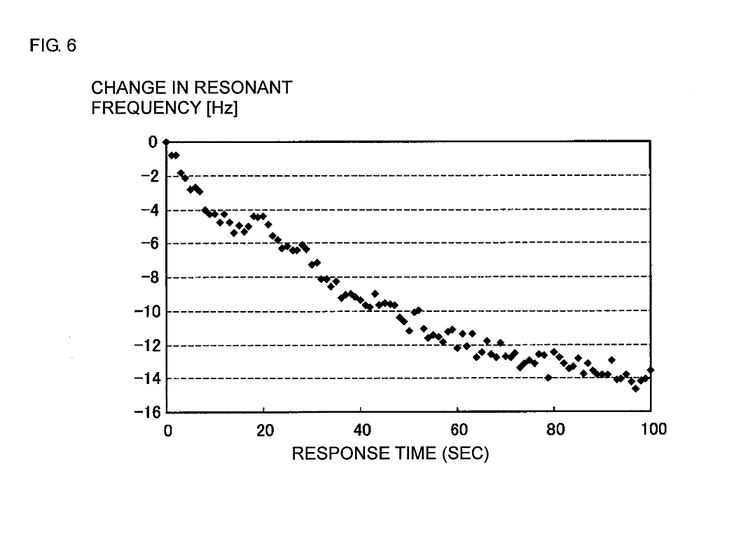

FIG. 6 shows the change in signal (resonant frequency) of the SAW device obtained when it was exposed to eugenol at a concentration of 0.1 mg/L. The horizontal axis indicates the time from the addition (response time), and the vertical axis indicates resonant frequency (change in resonant frequency).

It was found that when the SAW device was exposed to eugenol, an adsorption reaction occurred on the electrode surface, and the frequency was decreased.

Accordingly, it was shown that in the SAW device having a film formed of the polypeptide which was synthesized in such a way that by using the amino acid sequence of the olfactory receptor to eugenol, the transmembrane region, which was a part of the above amino acid sequence, was imitated, and an adsorption response to eugenol could be produced.

It was also shown that the piezoelectric oscillator was not limited to a quartz oscillator, and that a surface acoustic wave device having a film formed of a polypeptide was also able to respond to an odor substance.

The embodiment and the examples disclosed at this time are all described by way of example, and it is to be understood that the present invention is not limited thereto. The scope of the present invention is not shown by the above description but also includes equivalents and modifications.

### INDUSTRIAL APPLICABILITY

According to the present invention, a chemical sensor imitating the olfaction of living organisms can be manufactured as an electronic device, and olfactory sensation can be imparted, for example, to home electric appliances, such as a television and a cooking device, and communication apparatuses, such as a mobile phone, and that electrical appliance groups having new functionalities are developed and commercially produced.

When the present invention is applied to robots and the like, a robot which has functions similar to the olfaction of living organisms and which has sensations similar to that thereof can be produced.

1. A chemical sensor comprising a film containing at least one polypeptide which is or is homologous with at least a part of an amino acid sequence of an olfactory receptor.

2. The chemical sensor according to claim 1, wherein the film is disposed on a substrate.

3. The chemical sensor according to claim 2, wherein the substrate is a part of a piezoelectric device.

4. The chemical sensor according to claim 3, wherein the polypeptide interacts with a target molecule in a vapor phase, and the chemical sensor further comprises a signal sensor for receiving a signal resulting from an interaction between the polypeptide and the target molecule.

5. The chemical sensor according to claim 4, wherein the polypeptide specifically binds the target molecule.

6. The chemical sensor according to claim 1, wherein the polypeptide amino acid sequence is different from the amino acid sequence of another olfactory receptor at a corresponding portion thereof.

7. The chemical sensor according to claim 5, wherein the length of the polypeptide is equal to or less than the length of an α-helix region functioning as a transmembrane region of the amino acid sequence of the olfactory receptor.

8. The chemical sensor according to claim 5, wherein the polypeptide comprises at least two amino acid sequences each of which is or is homologous with portions of the amino acid sequence of the olfactory receptor which are not continuously arranged.

9. The chemical sensor according to claim 7, wherein the amino acid sequences of the polypeptides present are arranged so that the direction thereof from the N-terminus to the C-terminus substantially coincides with the corresponding direction in the olfactory receptor.

10. The chemical sensor according to claim 1, wherein the substrate is a part of a piezoelectric device.

11. The chemical sensor according to claim 10, wherein there are a plurality of amino acid sequences, each of which contains 4 to 35 amino acid residues, arranged to conform to the amino acid sequences in the α-helix regions of the olfactory receptor.

12. The chemical sensor according to claim 10, wherein the piezoelectric device is a quartz oscillator.

13. The chemical sensor according to claim 10,
wherein the piezoelectric device is an acoustic surface wave device.

14. The chemical sensor according to claim 1, wherein the chemical sensor further comprises a signal sensor for receiving a signal resulting from an interaction between the polypeptide and a target molecule.

15. The chemical sensor according to claim 14, wherein the polypeptide specifically binds target molecule.

16. The chemical sensor according to claim 1, wherein the length of the polypeptide is equal to or less than the length of an α-helix region functioning as a transmembrane region of the amino acid sequence of the olfactory receptor.

17. The chemical sensor according to claim 1, wherein the polypeptide comprises at least two amino acid sequences each of which is or is homologous with portions of the amino acid sequence of the olfactory receptor which are not continuously arranged.

18. The chemical sensor according to claim 1, wherein the polypeptide contains amino acid sequences arranged so that the direction thereof from the N-terminus to the C-terminus substantially coincides with the direction of the amino acid sequences in the olfactory receptor.

19. A method of forming a chemical sensor which comprises providing a polypeptide having at least one amino acid sequence which is or is homologous with a part of the amino acid sequence of an olfactory receptor, and forming a film thereof on a part of a piezoelectric device.

20. The method according to claim 19, wherein the amino acid sequences are or are homologous with the sequence of the α-helix regions of the olfactory receptor.

21. The method according to claim 20, wherein each amino acid sequence contains 4 to 35 residues of the α-helix regions of the olfactory receptor arranged to conform to the amino acid sequences of the olfactory receptor.


SEQUENCE LISTING (6 sequences)

SEQ ID NO: 1 - protein, 16 aa, Artificial Sequence
  Description of Artificial Sequence Synthetic peptide
LFVFATFNEISTLLIC

SEQ ID NO: 2 - protein, 13 aa, Artificial Sequence
  Description of Artificial Sequence Synthetic peptide
CITIFHGTILFLY

SEQ ID NO: 3 - protein, 15 aa, Mus musculus
LFVFATFNEISTLLI

SEQ ID NO: 4 - protein, 15 aa, Mus musculus
MGVANVIFLGVPVLF

SEQ ID NO: 5 - protein, 12 aa, Mus musculus
ITIFHGTILFLY

SEQ ID NO: 6 - protein, 12 aa, Mus musculus
VIIFYGTILFMY